SEQUENCE LISTING (5 sequences)

SEQ ID NO: 1 - DNA, 9029 bp, Homo sapiens
gcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagctgctcccagattactccttccttccaatataactaggcaaaaagaagtgaggagaaacctgcatgaaagcattcttccctgaaaagttaggcctctcagagtcaccacttcctctgttgtagaaaaactatgtgatgaaactttgaaaaagatatttatgatgttaacatttcaggttaagcctcatacgtttaaaataaaactctcagttgtttattatcctgatcaagcatggaacaaagcatgtttcaggatcagatcaatacaatcttggagtcaaaaggcaaatcatttggacaatctgcaaaatggagagaatacaataactactacagtaaagtctgtttctgcttccttacacatagatataattatgttatttagtcattatgaggggcacattcttatctccaaaactagcattcttaaactgagaattatagatggggttcaagaatccctaagtcccctgaaattatataaggcattctgtataaatgcaaatgtgcatttttctgacgagtgtccatagatataaagccatttggtcttaattctgaccaataaaaaaataagtcaggaggatgcaattgttgaaagctttgaaataaaataacaatgtcttcttgaaatttgtgatggccaagaaagaaaatgatgatgacattaggcttctaaaggacatacatttaatatttctgtggaaatatgaggaaaatccatggttatctgagataggagatacaaactttgtaattctaataatgcactcagtttactctctccctctactaatttcctgctgaaaataacacaacaaaaatgtaacaggggaaattatataccgtgactgaaaactagagtcctacttacatagttgaaatatcaaggaggtcagaagaaaattggactggtgaaaacagaaaaaacactccagtctgccatatcaccacacaataggatcccccttcttgccctccacccccataagattgtgaagggtttactgctccttccatctgcctgaccccttcactatgactacacagaatctcctgatagtaaagggggctggaggcaaggataagttatagagcagttggaggaagcatccaaagattgcaacccagggcaaatggaaaacaggagatcctaatatgaaagaaaaatggatcccaatctgagaaaaggcaaaagaatggctacttttttctatgctggagtattttctaataatcctgcttgacccttatctgacctctttggaaactataacatagctgtcacagtatagtcacaatccacaaatgatgcaggtgcaaatggtttatagccctgtgaagttcttaaagtttagaggctaacttacagaaatgaataagttgttttgttttatagcccggtagaggagttaaccccaaaggtgatatggttttatttcctgttatgtttaacttgataatcttattttggcattcttttcccattgactatatacatctctatttctcaaatgttcatggaactagctcttttattttcctgctggtttcttcagtaatgagttaaataaaacattgacacatac

SEQ ID NO: 2 - DNA, 2804 bp, Homo sapiens
accactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaaagatggatttccaaggttaattcattggaattgaaaattaacagggcctctcactaactaatcactttcccatcttttgttagatttgaatatatacattctatgatcattgctttttctctttacaggggagaatttcatattttacctgagcaaattgattagaaaatggaaccactagaggaatataatgtgttaggaaattacagtcatttctaagggcccagcccttgacaaaattgtgaagttaaattctccactctgtccatcagatactatggttctccactatggcaactaactcactcaattttccctccttagcagcattccatcttcccgatcttctttgcttctccaaccaaaacatcaatgtttattagttctgtatacagtacaggatctttggtctactctatcacaaggccagtaccacactcatgaagaaagaacacaggagtagctgagaggctaaaactcatcaaaaacactactccttttcctctaccctattcctcaatcttttaccttttccaaatcccaatccccaaatcagtttttctctttcttactccctctctcccttttaccctccatggtcgttaaaggagagatggggagcatcattctgttatacttctgtacacagttatacatgtctatcaaacccagacttgcttccatagtggagacttgcttttcagaacatagggatgaagtaaggtgcctgaaaagtttgggggaaaagtttctttcagagagttaagttattttatatatataatatatatataaaatatataatatacaatataaatatatagtgtgtgtgtgtatgcgtgtgtgtagacacacacgcatacacacatataatggaagcaataagccattctaagagcttgtatggttatggaggtctgactaggcatgatttcacgaaggcaagattggcatatcattgtaactaaaaaagctgacattgacccagacatattgtactctttctaaaaataataataataatgctaacagaaagaagagaaccgttcgtttgcaatctacagctagtagagactttgaggaagaattcaacagtgtgtcttcagcagtgttcagagccaagcaagaagttgaagttgcctagaccagaggacataagtatcatgtctcctttaactagcataccccgaagtggagaagggtgcagcaggctcaaaggcataagtcattccaatcagccaactaagttgtccttttctggtttcgtgttcaccatggaacattttgattatagttaatccttctatcttgaatcttctagagagttgctgaccaactgacgtatgtttccctttgtgaattaataaactggtgttctggttcat

SEQ ID NO: 3 - DNA, 1286 bp, Hepatitis B virus
gtcgagtataaaaacaatgagatctttgttgatcttggttttgtgtttcttgccattggctgctttgggtaaggttcgacaaggcatgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcaatggccagaggcaaatcaggtaggagcgggagcattcgggccagggttcaccccaccacacggcggtcttttggggtggagccctcaggctcagggcatattgacaacagtgccagcagcacctcctcctgcctccaccaatcggcagtcaggaagacagcctactcccatctctccacctctaagagacagtcatcctcaggccatgcagtggaattccacaacattccaccaagctctgctagatcccagagtgaggggcctatattttcctgctggtggctccagttccggaacagtaaaccctgttccgactactgcctcacccatatctggggaccctgcaccgaacatggagaacacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaaccaccagcacggggccatgcaagacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttaaattgaattgaattgaa

SEQ ID NO: 4 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

(19) United States
(12) Patent Application Publication
(10) Pub. No.: US 2003/0044982 A1
(43) Pub. Date: Mar. 6, 2003

(54) Method to treat hemophilia by hepatic gene transfer of factor VIII/IX with vesicle vector

(76) Inventors: Kenneth Chien, La Jolla, CA (US); Masahiko Hoshijima, La Jolla, CA (US)

Correspondence Address: BROWN, MARTIN, HALLER & MCCLAIN LLP, 1660 UNION STREET, SAN DIEGO, CA, 92101-2926, US

(21) Appl. No.: 10/132,829

(22) Filed: Apr. 25, 2002

Related U.S. Application Data
(60) Provisional application No. 60/286,314, filed on Apr. 25, 2001.

(51) Int. Cl. C12N 15/86; C12N 5/06

(52) U.S. Cl. 435/456; 435/325; 435/320.1

(57) ABSTRACT

Hemophilia is one of the most common genetic disorders. Standard therapies include transfusions with plasma products to provide clotting factors. The invention is a non-viral vesicle vector and method for the treatment of hemophilia. The vesicle vector contains the hepatitis B envelope protein to target the vesicle to the liver for delivery of an expression construct containing the coding sequence for factor VIII or IX driven by an appropriate promoter or factor VIII or IX protein.

### CROSS-REFERENCES TO RELATED APPLICATIONS

This application claims the benefit of priority of U.S. provisional application Serial No. 60/286,314 filed Apr. 25,2001 which is incorporated herein by reference in its entirety.

### SEQUENCE LISTING

A sequence listing is submitted herewith under 35 C.F.R. §1.821 and is incorporated herein by reference.

### BACKGROUND OF THE INVENTION

Hemophilia is one of the most common genetic disorders. Hemophilia A caused by deficiency of Factor VIII occurs in about 1 in 5000 male births, while hemophilia B caused by a defect in Factor IX is around 1 in 30,000 male births. The prevalence is very general in all populations studied. Hemophilia has long been treated with clotting factor concentrates, but the aim of this therapy is to control bleeding and requires lifelong repetitive intravenous infusions. Because of the increasing awareness of the risk of plasma derived products, the importance of the development of new and effective treatments is increased.

Gene therapy approaches have been developed for the treatment of hemophilia. Hemophilia is a particularly attractive model for developing a gene transfer approach for the treatment of disease. The proteins are well characterized, the genes are cloned and available, and there are large and small animal models of the disease. Moreover, there is no essential requirement for tissue specific delivery of the gene product and as protein function is regulated by activation of the protein; therefore, expression levels of the protein need not be tightly regulated. Additionally, only a low level of protein expression is required for phenotypic correction of the disease. The major hurdle of treatment of hemophilia by gene therapy is that the expression of the gene product must be sustained throughout the life of the individual; therefore, effective therapy would likely require re-administration of the gene therapy vector.

Clinical trials for the treatment of hemophilia using retroviral and adeno-associated viral (AAV) vectors are ongoing. Adenoviral and lentiviral vectors have been used experimentally. However, the problem with all of these viral vectors is that they have a limited capacity for nucleic acid and have been shown to elicit an immune response. The use of DNA or RNA with or without synthetic liposomes results in low efficiency gene transfer. Non-viral methods achieve only short term, non-targeted gene expression.

A novel, liver-specific vesicle vector expressing modified surface proteins of the hepatitis B virus was recently described by Yamada et al (2001 a). The vesicles containing the hepatitis B membrane proteins are generated by the methods well known to those skilled in the art (Kuroda et al,1992, and Yamada et al., 2001b, incorporated herein by reference). Briefly, a modified hepatitis B envelope (env) L protein, containing the pre-S1+pre-S2+S peptides, can be effectively generated in yeast by fusing the coding sequence for the chicken lysozyme signal sequence in frame to the beginning of the coding sequence for the modified env L protein (SEQ ID 1). The signal sequence directs the insertion of the proteins into the endoplasmic reticulum during translation. Protein rich vesicles bud from the endoplasmic reticulum and accumulate in the cytoplasm of the yeast cell. The vesicles are composed of lipid bilayers derived from the ER and the modified env L proteins as the major protein component. Particles formed by this method are very stable and can be easily purified through repetitive cesium chloride and sucrose gradients by methods well known to those skilled in the art.

Plasmid DNA can be incorporated into the env L containing particles by electroporation (Yamada et al. 2001a). Such DNA containing particles were demonstrated to facilitate entry of the DNA specifically into liver cells both in culture and upon systemic administration to nude mice in which human hepatoma cells were transplanted. Yamada et al. (2001a) suggested that such a vesicle vector could be used for tissue specific delivery of nucleic acid and other compounds to the liver.

### SUMMARY OF THE INVENTION

The invention is a non-viral vesicle vector for the treatment of hemophilia comprising a lipid bilayer containing a modified hepatitis B env L protein such that recognition of the S-peptide by the immune system is attenuated or abrogated, but the liver targeting signals are still exposed on the surface of the vesicle, and an expression construct for the expression of Factor VIII or IX for the treatment of hemophilia A or B, respectively. The expression construct may be single or double stranded DNA containing any of a number of promoters including, but not limited to general (e.g. cytomegalovirus, Rous sarcoma virus) and tissue specific (e.g. alpha fetoprotein, globulin, albumin, α1-microglobulin) promoters. The construct may contain additional regulatory elements including, but not limited to enhancers, introns, poly A sequences, RNA targeting sequences. Sequences to promote replication of the plasmid including SV40 origin of replication can be included. Inverted terminal repeat (ITR) sequences from AAV can be included in the construct to promote expression cassette stability or to enhance integration into the host DNA with the AAV Rep protein. In lieu of ITR sequences, eukaryotic DNA transposon/transposases systems can be used to promote integration.

The invention is a method for the treatment of hemophilia by administration of the non-viral vesicle vector of the invention. The vesicle vector containing the nucleic acid construct with the appropriate coding sequence is administered intravenously or intraarterially. The individual is monitored for expression of the gene product of interest by detection of the protein or mRNA or by phenotypic recovery.

### DETAILED DESCRIPTION AND PREFERRED EMBODIMENT

Hemophilia is one of the most common genetic disorders and is a result of a mutation or deletion in any of the clotting factors, most commonly Factor VIII or IX. Treatment requires the lifelong replacement of clotting factors which requires repetitive intravenous infusions and exposes patients to the dangers associated with plasma derived products.

Hemophilia is amenable to treatment with gene therapy for a number of reasons. First, the genes involved are cloned and available. Second, the proteins are well characterized and their activation is regulated by cleavage of the protein rather than at the transcriptional or translational level; therefore, the expression level does not need to be tightly regulated. Third, low levels of protein expression have been demonstrated to be sufficient for phenotypic recovery. Fourth, although the liver is the physiological site of production of most of the Factor VIII and IX, the site of production of the protein within the body is relatively unimportant. Fifth, a number of animal models are available for analysis of various therapies. However, to date no effective gene transfer vectors or methods for the treatment of hemophilia have been developed.

The invention is a vesicle vector for the treatment of hemophilia comprising a natural lipid vesicle preferably produced in yeast or insect cells, such as Sf9 cells, containing modified hepatitis B env L protein integrated into the membrane and an expression construct inside the vesicle for the expression of Factor VIII or IX. The vesicles are prepared by the vaccine production method of Kuroda (1992) further refined by Yamada (2001b). Briefly, the hepatitis B env L protein is composed of three regions: the 108- or 119-residue pre-S1 region involved in the direct interaction with hepatocytes, the 55-residue pre-S2 region associated with the polymerized albumin-mediated interaction and the major 226-residue S-protein region. Attempts to produce L protein in various eukaryotic cells had been unsuccessful, probably due to the presence of the N-terminus of the pre-S1 peptide. The coding sequence of the N-terminus of the L protein was replaced by a chicken lysosome signal sequence to direct the translocation of the N-terminus through the endoplasmic reticulum (ER). The chimeric sequence was inserted into a yeast (S. cerevisiae) expression vector and inserted into yeast using a standard transformation protocol. The chimeric L-protein was produced in abundance, up to 42% of the total yeast protein, and was properly inserted into the membrane. Vesicles budded off of the ER to form 23 nm spherical and filamentous particles containing the protein in the membrane of the vesicles. The yeast cells were disrupted with glass beads to release the vesicles. Vesicles were purified by serial rounds of discontinuous cesium and sucrose gradients. Production and purification of vesicles from insect cells would be performed in a similar method. A crude membrane fraction could be prepared as with the yeast cells, by homogenization and differential centrifugation. The fraction can be loaded onto cesium or sucrose gradients as with the yeast extract for purification of vesicles. The methods are amenable to inexpensive, large scale production of vesicles which is necessary for gene transfer. Vesicles are stable for long term storage at a low temperature but are unstable upon repeated freeze-thaw cycles.

The vesicle vectors can be used for the delivery of any nucleic acid construct, single- or double-stranded DNA or RNA, or gene product to the liver. In a preferred embodiment of the invention, the nucleic acid is a double stranded DNA plasmid. The construct minimally contains the coding sequence for human Factor VIII (SEQ ID 2) or IX (SEQ ID 3) for the treatment of hemophilia A or B respectively and a promoter to allow for transcription of the hemophilia gene. The construct may optionally contain additional regulatory and enhancer elements to modulate gene expression, intron and poly-A sequences to promote RNA processing and gene expression, RNA targeting sequences, AAV-ITR or eukaryotic transposon sequences to promote stabilization of expression cassettes and integration into the host genome and viral origin of replication sequences to promote amplification of the plasmid in host cells. Such sequences are well known to those skilled in the art. The number of elements that can be inserted into the nucleic acid construct as the size is not limited by the requirements of a viral genome as is the case with many gene transfer protocols.

Any of a number of promoter sequences are known to be functional in liver cells. These include both non-tissue specific promoters such as CMV, RSV, ubiquitin, chicken β-actin and elongation factor (EF)-1α; and tissue specific promoters such as alpha-fetoprotein, globulin, α1-microglobulin and albumin.

AAV-ITR sequences may be incorporated into the construct flanking all of the coding and regulatory sequences, other than any origins of replication. The AAV-ITR sequences have been demonstrated to increase the stability of transferred constructs in gene therapy protocols. Alternatively, the AAV-ITR sequences may enhance integration into the human genome at a specific site with the cooperation of the AAV-Rep protein, which may be supplied by incorporation into the vesicles with the nucleic acid construct or by expression cassettes packaged into the same vesicle.

Eukaryotic transposon sequences can be incorporated into the construct flanking all of the coding sequences and regulatory elements, similar to the AAV-ITR sequences. Transposase to promote integration may be expressed from the same expression cassette or from a separate expression cassette packaged into the same vesicle.

Special considerations may be taken when expressing Factor VIII. Studies have demonstrated that human Factor VIII contains a sequence (nulceotides 1741 to 1771 in SEQ ID 2) that decreases heterologous expression of proteins (Fallaux et al., 1996). The sequence is AT-rich and has been demonstrated to bind a nuclear factor and repress expression of a reporter construct in cells. Deletion or random mutation of the sequence results in a non-functional Factor VIII. However, silent mutations that result in no change in the amino acid sequence of the gene product can be introduced into the coding sequence by methods well known to those skilled in the art to enhance expression of Factor VIII.

In a preferred embodiment, the nucleic acid construct of the invention is introduced into the vesicles by electroporation. The nucleic acid construct is mixed thoroughly with the vesicles, brought to a final volume in water and transferred to an electroporation cuvette. Voltage and resistance vary widely depending on the size (gap length) of the cuvette and the volume of material in the cuvette. Such parameters can be readily modified by methods well known to those skilled in the art to result in maximum transfer of nucleic acid into vesicles with minimum destruction of vesicles.

Alternatively the nucleic acid may be introduced into the vesicle by fusion with nucleic acid containing liposomes by methods well known to those skilled in the art (Dzau et al, 1996). The construct of the invention is encapsulated into liposomes prepared by vortexing. Liposomes may be composed of known phospholipids and membrane components (e.g. phosphatidyl-choline, cholesterol) or of commercially available proprietary mixtures of membrane components (e.g. Lipofectamine from Gibco-BRL). Nucleic acid encapsulated in liposomes will fuse with the yeast or insect cell derived vesicles upon incubation at 37° C. for 10-30 minutes.

Alternatively, factor VIII or IX protein may be incorporated into the vesicle vector of the invention. Factor VIII (SEQ ID 4) and IX (SEQ ID 5) protein may be produced using any of a number of methods well known to those skilled in the art. A solution containing a high concentration of protein may be mixed with purified vesicles and subjected to osmotic shock or sonication to promote incorporation of the protein into the vesicles. Protein may also be incorporated into artificial membranes by vortexing or sonication. The artificial membranes containing the protein can be fused with the hepatitis B vesicles.

The nucleic acid or protein containing non-viral vesicle vectors of the invention are administered to the individual intravenously or intraarterially. To increase delivery, the vesicle vector can be administered directly into the hepatic or portal artery. The individual is monitored on regular intervals for the presence of factor VIII or IX or for phenotypic recovery. The amount of the non-viral vesicle to be administered would depend on the strength of the promoter, integration sequences, number of plasmids per vesicle and a number of other considerations well know to those skilled in the art. As methods for monitoring the state of health of individuals are well known, an effective dose can be readily determined.

Although an exemplary embodiment of the invention has been described above by way of example only, it will be understood by those skilled in the field that modifications may be made to the disclosed embodiment without departing from the scope of the invention, which is defined by the appended claims.

### REFERENCES

Fallaux, F. J. et al (1996) The human clotting factor VIII cDNA contains an autonomously replicating sequence consensus- and matrix attachment region-like sequence that binds a nuclear factor, represses heterologous gene expression, and mediates the transcriptional effects of sodium butyrate. Mol. Cell. Biol. 16:4264-4272.

Kuroda, S. et al (1992) Hepatitis B virus envelope L protein particles. J. Biol. Chem. 267:1953-1961.

Yamada, T. et al (2001a) A new pinpoint gene delivery system using genetically engineered hepatitis B virus envelope L particles. Molecular Biology and New Therapeutic Strategies: Cancer Research in the 21^{st} Century. 5^{th} Joint Conference of the American Association for Cancer Research and the Japanese Cancer Association. Hawaii, USA, Feb. 12-16, 2001.

Yamada. T. et al (2001b) Physiochemical and immunological characterization of hepatitis B virus envelope particles exclusively consisting of the entire L (pre-S1+pre- S2+S) protein. Vaccine 19:3154-3163.

We claim:

1. A non-viral vesicle vector comprising:
a vesicular membrane with hepatitis B envelope (env) protein exposed on the vesicle surface and
a nucleic acid expression construct comprising a complete factor VIII or factor IX coding sequence and a promoter sequence functional in liver cells.

2. The vesicle vector of claim 1, wherein the env protein contains mutations to reduce antigenicity.

3. The vesicle vector of claim 1, wherein the expression construct is DNA.

4. The vesicle vector of claim 1, wherein the expression construct is double stranded plasmid DNA.

5. The vesicle vector of claim 1, wherein the expression construct is RNA.

6. The vesicle vector of claim 1, wherein the promoter is a non-tissue specific promoter.

7. The vesicle vector of claim 6, wherein the non-tissue specific promoter is selected from the group consisting of cytomegalovirus promoter, Rous sarcoma virus promoter, ubiquitin promoter, chicken β-actin promoter and elongation factor 1α promoter.

8. The vesicle vector of claim 1, wherein the promoter is a liver specific promoter.

9. The vesicle vector of claim 8, wherein the liver specific promoter is selected from the group consisting of alpha-fetoprotein promoter, globulin promoter, α1-microglobulin and albumin.

10. The vesicle vector of claim 1, wherein the expression construct comprises inverted terminal repeat sequences from adeno-associated virus (AAV-ITR).

11. The vesicle vector of claim 1, wherein the expression construct comprises eukaryotic transposon and transposase sequences.

12. The vesicle vector of claim 1, wherein the expression construct comprises the coding sequence of factor VIII.

13. The vesicle vector of claim 12, wherein the factor VIII comprises silent mutations to enhance expression.

14. The vesicle vector of claim 1, wherein the expression construct comprises the coding sequence of factor IX.

15. A non-viral vesicle vector comprising:
a vesicular membrane with hepatitis B envelope (env) protein exposed on the vesicle surface and
a protein comprising a complete factor VIII or factor IX.

16. The vesicle vector of claim 15, wherein the env protein contains mutations to reduce antigenicity.

17. A method for treatment of hemophilia comprising:
administration into circulation of an individual with hemophilia a non-viral vesicle vector comprising a vesicular membrane with hepatitis B env protein exposed on the vesicle surface and
a nucleic acid expression construct comprising a complete factor VIII or IX coding sequence and a promoter sequence functional in liver cells and
monitoring the individual for amelioration of disease.

18. The method of claim 17, wherein administration into circulation comprises intravenous administration.

19. The method of claim 17, wherein administration into circulation comprises administration into a hepatic or portal artery.
